(19) United States
(12) Patent Application Publication
(10) Pub. No.: US 2024/0197637 A1
(43) Pub. Date: Jun. 20, 2024

(54) POLYMERIC MICROPARTICLES FOR THE LOCAL TREATMENT OF CHRONIC INFLAMMATORY DISEASES

(71) Applicants: FONDAZIONE ISTITUTO ITALIANO DI TECNOLOGIA, Genova (IT); The University of North Carolina at Chapel Hill, Chapel Hill, NC (US)

(72) Inventors: Paolo DECUZZI, Genova (IT); Martina DI FRANCESCO, Genova (IT); Lara LONGOBARDI, Chapel Hill, NC (US); Huseyin OZKAN, Chapel Hill, NC (US)

(21) Appl. No.: 18/287,093

(22) PCT Filed: Apr. 13, 2022
(86) PCT No.: PCT/EP2022/059960

§ 371 Date: Oct. 16, 2023

(30) Foreign Application Priority Data
Apr. 16, 2021 (IT) 102021000009632

(51) Int. Cl. A61K 9/16; A61K 31/537; A61K 31/573; A61K 31/7105; A61K 45/06; A61P 19/02

(52) U.S. Cl. CPC A61K 9/1647; A61K 31/537; A61K 31/573; A61K 31/7105; A61K 45/06; A61P 19/02

(57) ABSTRACT

The present invention relates to drug-delivery compositions comprising microparticles with the shape of a prism comprising at least one polymer and at least one CCR2 inhibitor. Preferably said composition is for the treatment of local chronic inflammation diseases, such as osteoarthritis.

### FIELD OF THE INVENTION

The present invention relates to the field of the local treatment of chronic inflammatory diseases, in particular osteoarthritis.

In particular, it relates to drug-delivery compositions for local administration comprising polymeric microparticles.

### BACKGROUND OF THE INVENTION

Osteoarthritis (OA) is a chronic inflammatory joint disease that affects elderly population worldwide. Moreover, nearly half of the subjects experiencing a significant damage to ligaments, menisci, or articular surfaces will develop OA. The Global Burden of Disease Study reports that 240 million people aged over 60 have some degree of OA, with higher prevalence in women (18% women vs 10% men) (Nelson, A. E., Osteoarthritis year in review 2017: clinical. Osteoarthritis and cartilage, 2018. 26(3): p. 319-325).

Osteoarthritis has all the hallmarks to be considered one of the most prevalent worldwide disease, with a tremendous symptomatic and economic global burden. In addition, OA is regarded as the main cause of permanent disability and the third cause of temporary workplace incapacity. Almost any joint can be affected by OA, but knees, hips and small joints of the hands are the most affected.

At present, there is no drug that can prevent, arrest, or even restrain OA progression. Drugs available on the market aim only to alleviate symptoms.

Several pharmacological and non-pharmacologic approaches, either individually or in combination, are applied for OA management (Loeser, R. F., J. A. Collins, and B. O. Diekman, Ageing and the pathogenesis of osteoarthritis. Nature Reviews Rheumatology, 2016. 12(7): p. 412-420). From a pharmacological standpoint, small molecules, such as acetaminophen (paracetamol), non-steroidal anti-inflammatory drugs (NSAIDs), opioid analgesics or COX-2 inhibitors are the most used. In particular, the intra-articular injection of NSAIDs is one of the first line in OA treatment (Grassel, S. and D. Muschter, Recent advances in the treatment of osteoarthritis. F1000Research, 2020. 9).

Because of the OA localized nature, the direct delivery into affected joints represents an attractive alternative to systemic delivery for both maximizing drug bioavailability at the target site and reducing systemic exposure. Biomaterials could be used to develop depots able to improve drug pharmacokinetics, especially in case of chronic diseases. These nano- and micro-particles, nanofibers, and hydrogels have been extensively tested in preclinical and clinical drug delivery formulations for OA treatment (Kou, L., et al., Biomaterial-engineered intra-articular drug delivery systems for osteoarthritis therapy. Drug delivery, 2019. 26(1): p. 870-885). On this attempt, Food and Drug Administration (FDA) has approved in October 2017 Zilretta, poly(D,L-lactide-co glycolide) acid (PLGA, 75:25 molar ratio lactide to glycolide) microparticles for extended-release of triamcinolone acetonide, a corticosteroid, for intra-articular therapy of OA knee pain. This is the first long-acting formulation approved for the intra-articular OA knee pain management. It has been developed to stay in the knee joint, promoting the continuous and sustained release of drug in the target site over a period of 12 weeks, thus reducing its clearance and its systemic side effects (Bodick, N., et al., Corticosteroids for the treatment of joint pain. 2014, WO2012019009A1). Unfortunately, this acts only on the pain management on both the Average Daily Pain (ADP) and Western Ontario and McMaster Universities Arthritis Index (WOMAC, pain) scales (Paik, J., S. T. Duggan, and S. J. Keam, Triamcinolone acetonide extended-release: a review in osteoarthritis pain of the knee. Drugs, 2019. 79(4): p. 455-462).

In the last years, several experimental data are supporting the notion that inflammatory chemokines can actively alter the joint tissue metabolism in OA. In fact, they promote cell migration towards sites of inflammation and induce OA progression (Miller, R. E. and A.-M. Malfait, Can we target CCR2 to treat osteoarthritis? The trick is in the timing! Osteoarthritis and cartilage, 2017. 25(6): p. 799-801; Yuan, G. H., et al., The role of C-C chemokines and their receptors in osteoarthritis. Arthritis & Rheumatism: Official Journal of the American College of Rheumatology, 2001. 44(5): p. 1056-1070).

Higher baseline serum levels of chemokine (C-C motif) ligand 2 (CCL2) are detected in the blood, synovial fluid and synovial tissue in OA patients and are connected with progression of radiographic OA at 5-year follow up (Longobardi, L., et al., Associations between the chemokine biomarker CCL2 and knee osteoarthritis outcomes: the Johnston County Osteoarthritis Project. Osteoarthritis and cartilage, 2018. 26(9): p. 1257-1261). CCL2 demonstrated chondrocyte degradation, while CCL2/CCR2 mediates OA pain.

C-C Chemokine Receptor-2 (CCR2) is a receptor recognized as an important potential target in OA. CCR2 is presented in sensory neurons, and the CCL2 interaction can directly excite nociceptive neurons, thereby contributing to pain. Several studies demonstrated that CCR2-/-animals were more sensitive to pain-related behavior (decreases in climbing and locomotion) compared to in wild type (WT), but no differences in chondropathy scores between the 2 groups were reported (Miller, R. E., et al., CCR2 chemokine receptor signaling mediates pain in experimental osteoarthritis. Proceedings of the National Academy of Sciences, 2012. 109(50): p. 20602-20607; Zarebska, J. M., et al., CCL2 and CCR2 regulate pain-related behaviour and early gene expression in post-traumatic murine osteoarthritis but contribute little to chondropathy. Osteoarthritis and cartilage, 2017. 25(3): p. 406-412; Raghu, H., et al., CCL2/CCR2, but not CCL5/CCR5, mediates monocyte recruitment, inflammation and cartilage destruction in osteoarthritis. Annals of the rheumatic diseases, 2017. 76(5): p. 914-922).

Longobardi and coworkers have demonstrated that the levels of CCL12 (homologue of human CCL2, a CCR2 ligand) increased in articular cartilage, periosteum and osteoblasts in the early OA stage. They also proved that a highly specific CCR2 inhibitor, RS 504393, orally and daily administered, decreased the severity of structural damage in the DMM murine model of injury-induced OA as well as pain perception, by inhibiting macrophage recruitment to the dorsal root ganglion (Raghu, H., et al., CCL2/CCR2, but not CCL5/CCR5, mediates monocyte recruitment, inflammation and cartilage destruction in osteoarthritis. Annals of the rheumatic diseases, 2017. 76(5): p. 914-922; Longobardi, L., et al., Role of the CC chemokine receptor-2 in a murine model of injury-induced osteoarthritis. Osteoarthritis and cartilage, 2017. 25(6): p. 914-925). These results highlight the potential efficacy of antagonizing CCR2 at early stages during OA progression and at short time to prevent, or reduce, the development of cartilage and bone changes as well as pain.

However, a prolonged and delayed exposure to the RS504393 antagonist did not translate in a significant reduction of cartilage and bone damage, although alleviating pain (Longobardi et al., 2017).

Several studies conducted in inflammatory arthritis models using the CCR2 null mice have suggested that lack of CCR2 was accompanied by a decreased number of anti-inflammatory T regulatory cells, suggesting that CCR2 expression in a specific hematopoietic cell compartment may serve as negative regulator of arthritic disease severity. Therefore, although reducing the damage in cartilage and bone, a delayed and prolonged systemic CCR2 blockade at the more severe OA stages, when the inflammatory response is higher, might enhance inflammation, affecting OA progression and obstructing the beneficial action seen in the cartilage and bone compartment.

Therefore, there is still the need of an efficient system to administer drugs for the long-term treatment of osteoarthritis (OA). In particular, there is the need of a system which avoids the systemic effects of a prolonged anti-inflammatory drugs administration, more in particular the effects of a prolonged blockade of CCR2.

WO2020163871 discloses a drug-delivery composition for ocular administration through injection in the eye of a subject which comprises a capsule having a bi-layered wall and a therapeutic agent, wherein the therapeutic agent is initially present within a luminal space of the capsule. The capsule has a tubular shape and a macroscopic size and is not suitable for intra-articularly injection. The composition is indeed intended for the treatment of ophthalmological disorders.

WO2019200181 discloses compositions for drug delivery to an eye which can comprise particles having a core component comprising a first polymer, such as chitosan, and one or more therapeutic agents, and a shell layer comprising a second, biodegradable, polymer. Particles can be essentially spheres, spheroids or ellipsoids.

WO2010017265 discloses microspheres for intra-articular administration to the joint of a patient which may contain treatment agents and may be of various shapes.

In the work “Engineering shape-defined PLGA microPlates for the sustained release of anti-inflammatory molecules.” (Journal of Controlled Release 319 (2020) 201-212), Di Francesco et al. describe shape-defined poly(D,L-lactide-co-glycolide) (PLGA) microPlates (μPLs) realized for the sustained release of two anti-inflammatory molecules, the natural polyphenol curcumin (CURC) and the corticosteroid dexamethasone (DEX). The anti-inflammatory activity of such microplates was tested in vitro on rat monocytes and in vivo on a murine model of UVB-induced skin burns, wherein microplates loaded with curcumin were applied topically.

### SUMMARY OF THE INVENTION

It has now been found that polymer-squared based microparticles (μPLs) loaded with CCR2 inhibitors provide a sustained release of the loaded therapeutic agent which is efficient both at the early and severe osteoarthritis stages, without interfering with the systemic inflammatory response.

It is an object of the invention a drug-delivery composition comprising one or more microparticles, each microparticle having a non spherical shape, namely a prism shape with three or more sides, each side with a side length comprised between 5 and 50 μm and a height length comprised between 5 and 50 μm, and comprising at least one polymer and at least one CCR2 inhibitor.

The use of this composition offers many advantages over the free drug administration of CCR2 inhibitor, in particular:
it addresses the low solubility of the compound in a physiological solution;
it allows a local and sustained delivery within the diseased tissue, avoiding the side effects connected with systemic and daily administration of the CCR2 inhibitor;
it enhances the beneficial effect on cartilage and bone structure, at both the early and late OA stages, combining pharmacological and mechanical effects.

Indeed, it has been found that such microparticles when injected locally in the joint provide a sustained release of the loaded therapeutic agents for several weeks after a single injection, thus improving patient's compliance, limiting side effects and boosting therapeutic efficacy. This together with the unique mechanical features of the particles allow to create an intra-articular depot that promotes the continuous inhibition of the CCR2 receptor in the joint of a subject with OA. The localized drug release allows to avoid the systemic inhibition of CCR2 that is accompanied by the number reduction of anti-inflammatory T regulatory cells, which might enhance inflammation, affecting OA progression and obstructing the beneficial action seen in the cartilage and bone compartment.

The composition of the invention is therefore a long-acting formulation that can be used for stopping and restraining OA progression or other chronic pathologies that currently require drug daily administration.

It has now been found by the inventors that the continuous release of the therapeutic in the target is reflected on the in vivo efficacy on both cartilage and bone structure at both the early and late OA stages in a murine model of destabilization of medial meniscus (DMM). Notably, such effect in vivo was not achieved by the oral administration of the free drug, where prolonged CCR2 blockade produced no significant differences in cartilage and bone structure at the most severe OA stages.

Finally, the drug delivery composition of the invention has the advantage to allow to modulate drug release profile changing some features of the microparticles, such as the height and the amount of polymer used.

The drug-delivery composition of the invention for use as a medicament is also an object of the invention.

The drug-delivery composition of the invention for use for the prevention and/or treatment of a localized chronic inflammation disease, in particular osteoarthritis, is a further object of the invention.

A pharmaceutical composition comprising the drug delivery composition of the invention together with a pharmaceutically acceptable excipient and/or carrier is also an object of the invention.

Embodiments and experiments illustrating the principles of the invention will be discussed with reference to the following figures.

### BRIEF DESCRIPTION OF THE FIGURES

FIG. 1. μPLs physico-chemical characterization. A. SEM images of the empty PVA template; B. SEM image of μPLs released (20×20×10 μm) from the PVA template; C. Size characterization of μPLs via Multisizer analysis.

FIG. 2. Drug release. RS 504393 release profile from μPLs under confined conditions, mimicking the synovial volume (500 μL).

FIG. 3. In vivo efficacy of RS 504393-loaded μPLs in the destabilization of medial meniscus (DMM) murine model of post-traumatic OA (PTOA) on (A) cartilage structure (Articular cartilage score, ACS, 1-12 scale) and extracellular matrix composition (Saf-O Score, 1-12 scale, as well as on (B) osteophyte size and maturity (tissue composition as amount of cartilage content, 1-3 scale).

FIG. 4. Mechanical properties of μPLs. a. Force-displacement curve for a flat punch indentation experiment on μPLs (average curve and standard deviation). In the inset, a schematic of the experimental setup is provided; b. Mechanical damping of μPLs upon cyclic loading (frequency 5 Hz) as a function of the force oscillation amplitude. In the inset, a schematic of the testing routine highlights the phase angle d—dissipation parameter. Results are presented as the average±SD (n=3).

FIG. 5: μPLs interaction with different cell lines. (a) 30° tilted view of a SEM image of ATDC5 cells (chondrocytes) incubated with μPLs. In the lateral inset, a magnified image shows cells interacting with μPLs; (b) False-color SEM image of a μPL deposited and not internalized over a layer of ATDC5 cells; (c) SEM images of RAW 264.7 cells (macrophages) incubated with μPLs. The lateral inset shows a magnified image of cells surrounding a μPL without internalizing it; (d) false-color SEM image of μPLs surrounded by RAW 264.7 macrophages.

FIG. 6: In vivo pharmacokinetic study of Cy5-conjugated μPLs (Cy5-μPLs) in a PTOA mouse model. (a) Representative pharmacokinetic time course intravital images (skin on) and ex vivo knee images (skin off) of Cy5-μPLs injected intra-articularly into PTOA mouse knee joints (D-#, where #represents days after intra-articular injection); (b) Intravital fraction of retention of Cy5-μPLs plotted as mean+standard error; (c) Anatomically labeled sagittal section of a mouse knee joint 1 day after intra-articular injection showing the Cy5-μPLs dispersed across the joint interacting and/or in close proximity to many different tissue types such as the cartilage, the infrapatellar fat pad and synovium, and the joint capsule; (d) Confocal microscopy imaging performed 1 day after intra-articular injection showing Cy5-μPLs located on top of the cartilage surface, near the cartilage/synovium interface, and the joint capsule. In all images, the scale bar=100 m; (e) Confocal microscopy imaging of Cy5-μPLs within the mouse knee joint taken at different time points after intra-articular injection. TD=transmission detector. NT=no treatment. For intravital imaging analysis, n=4-24 limbs depending on the time point, that is, earlier time points had more animals included, and the sample size at the later time points was lower because some animals were taken down at earlier time points for ex vivo and confocal microscopy analysis. For ex vivo imaging analysis and confocal microscopy analysis, n=2-4 limbs per time point.

### DETAILED DESCRIPTION OF THE INVENTION

For drug delivery composition is herein intended a composition suitable for the delivery of one or more drugs.

For drug is herein intended any substance that causes a change in an organism's physiology or psychology.

For microparticle is herein intended a particle between 1 and 100 μm in size.

In an embodiment, the drug delivery composition of the invention is suitable for localized delivery to the joint of a subject, in particular a subject with a localized chronic inflammation disease, preferably osteoarthritis.

In an embodiment, the drug delivery composition of the invention is suitable for intra-articular injection.

The drug delivery composition of the invention comprises one or more microparticles. Usually, it comprises at least 100 or at least 1000 microparticles.

Each microparticle has a non-spherical shape, namely the shape of a prism. For prism it is intended a polyhedron comprising a first n-sided polygonal base, a second base which is a translated copy, i.e. rigidly moved without rotation, of the first, and n other faces joining corresponding sides of the two bases. The other faces are all parallelograms. n is the number of sides and it can vary between 3 and 8, preferably it is 4.

Indeed in a preferred embodiment, the microparticle has the shape of a prism with a square base.

The non-spherical shape of the microparticles advantageously facilitates their adhesion to the cartilage and synovial surfaces improving the delivery of the therapeutic agents towards the diseased tissue and also protects the cartilage surface from further mechanical abrasion and erosion.

Also, in the treatment of osteoarthritis, therapeutic agents need to be delivered to macrophages and non-phagocytic cells (chondrocytes); particles may be uptaken by macrophages and consequently degraded and metabolized, so that their therapeutic function is permanently loss.

The inventors demonstrated the ability of microparticles according to the invention to resist macrophage uptake and adhere to the cartilaginous tissue. Indeed, microparticles are not internalized by chondrocytes and macrophages, thus remaining extracellularly in the synovial cavity and promoting drug release and mechanical support at the target site. Also, microparticles of the invention are able to deform under mechanical loading.

Furthermore, microparticles according to the invention are able to be intra-articularly retained when injected, thus advantageously exercising their therapeutic action on the target site and not systemically.

Collectively, these features render the microparticles particularly advantageous to deliver a CCR inhibitor in a localized and sustained manner.

Each side of the microparticle has a side length comprised between 5 and 50 μm, preferably comprised between 10 and 20 μm, more preferably it is of 20 μm In a preferred embodiment, the microparticle has a square base with a side length comprised between 5 and 50 μm, preferably comprised between 10 and 20 μm, more preferably it is of 20 μm.

The height length of each side of the microparticle is comprised between 5 and 50 μm, preferably is comprised between 5 and 10 μm, more preferably it is of 10 μm.

In a preferred embodiment, the particle has a square base with a side length of 20 μm and a height of 10 μm, i.e. it is a 20 μm×20 μm×10 μm particle.

Each microparticle can have a Young's modulus under compression comprised between 1 KPa and 10 MPa, preferably from 100 KPa to 10 MPa.

The microparticles comprised in the drug-delivery composition of the invention are herein also named microplates or μPLs.

The flexibility of the microparticle can be finely tuned by changing its geometry and polymer content to favor the integration with the surrounding biomedical environment. In particular, soft conforming particles laying along the cartilage surface can be useful to prevent abrasion, whereas more rigid particles dispersed within the synovial fluid can be useful to enhance the biomechanical properties of the overall joint. The microparticle size and shape and mechanical properties can be finely tuned based on the specific applications and biomedical aim.

The skilled in the art is able to decide the suitable features in view of the above information and of the general knowledge in the field, see in particular Di Francesco et al., 2018 (ACS Appl Mater Interfaces. 2018 Mar. 21; 10(11):9280-9289. doi: 10.1021/acsami.7b19136. Hierarchical Microplates as Drug Depots with Controlled Geometry, Rigidity, and Therapeutic Efficacy) and Di Francesco et al. 2020, mentioned above.

The composition of the invention can be obtained with a method comprising the following steps:
a. synthesis of a silicon master template. This can be typically done using direct laser writing. The template allows to define a pattern of wells of the desired geometry and size. Wells size and shape correspond to the final geometry of the microparticles.
b. Deposition of poly(dimethylsiloxane) (PDMS) on the silicon template micropattern, which is then cured using a conventional ratio, for example 1:10 v/v, between the curing agent and the elastomer. Curing conditions are as usual in the field, for example 60° C. for 4 hours. This first intermediate template presents the opposite configuration of the silicon template, i.e. pillars instead of wells.
c. Spreading of a polymer solution on the first PDMS intermediate template and promotion of water evaporation, for example maintaining the template at 60° C. The obtained second intermediate polymer template shows the geometrical pattern of the original silicon master template, i.e. wells with same size and shape.
d. Spreading on the second intermediate polymer template obtained in the previous step of a solution of the polymer and of a solution of the CCR2 inhibitor which are to be comprised in the microparticle.
e. After solvent evaporation, dissolution of the second intermediate polymer template in water and collection of the purified microparticles.

In step c) the polymer solution which is spread on the first template typically comprises the polymer in a percentage w/v comprised between 2.5 and 10% w/v. The polymer used in step c) can be for example Poly(vinyl alcohol) (PVA), gelatin or agarose. Other polymers may be suitable, PVA is preferred.

The polymer comprised in the microparticle and used in step d) above can be selected from biocompatible hydrophobic or hydrophilic, synthetic or natural polymers. For example it can be selected from poly(lactic-co-glycolic acid)(PLGA), polyethylene glycol (PEG), polycaprolactone (PCL), hyaluronic acid (HA), chitosan, gelatin or a combination thereof. Preferably it is the hydrophobic polymer poly(lactic-co-glycolic acid)(PLGA). PLGA can comprise any lactide to glycolide molar ratio, 50:50 is a preferred ratio. Different polymer density and molecular weight can be used for tuning the mechanical stiffness of the resulting microparticles, based on their medical application. Also, the polymer mass can influence the drug release profile. The amount of polymer used in step d) can be comprised between 1 and 20 mg for each template, for example 15 mg. The polymer is usually dissolved in a suitable solvent, which the skilled person can select according to the general knowledge in the field. For example PLGA can be solubilized in acetonitrile. Concentration of the polymer when used in step d) can be comprised between 100 and 500 mg/ml, for example 400 mg/ml.

The CCR2 inhibitor can be selected from RS504393, Maraviroc, cenicriviroc, CD192, CCX872, CCX140, 2-((Isopropylaminocarbonyl)amino)-N-(2-((cis-2-((4(methylthio)benzoyl)amino)cyclohexyl)amino)-2-oxoethyl)-5-(trifluoromethyl)-benzamide, vicriviroc, SCH351125, TAK779, CCR2 antagonist 4 hydrochloride (Teijin compound 1 hydrochloride). Preferably it is RS504393. The CCR2 inhibitor is usually dissolved in a suitable solvent, for example RS504393 can be solubilized in acetonitrile. Concentration of the CCR2 inhibitor when used in step d) can be comprised between 0.5 and 2 mg/mL.

For CCR2 inhibitor it is intended a compound able to inhibit, at least partially, the C-C chemokine receptor type 2 (CCR2). Typically it is a compound which binds to said receptor and decreases its activity. Any compound able to inhibit the C-C chemokine receptor type 2 (CCR2) can be used in the microparticle of the invention. Preferred compounds are mentioned above. The skilled person is able to determine if a compound is an inhibitor of CCR2 and to find suitable CCR2 inhibitors based on the common general knowledge in the field, see for example Furuichi K, Wada T, Iwata Y, Kitagawa K, Kobayashi K, Hashimoto H, et al. Gene therapy expressing amino-terminal truncated monocyte chemoattractant protein-1 prevents renal ischemia-reperfusion injury. J Am Soc Nephrol 2003; 14(4):1066e71; Kitagawa K, Wada T, Furuichi K, Hashimoto H, Ishiwata Y, Asano M, et al. Blockade of CCR2 ameliorates progressive fibrosis in kidney. Am J Pathol 2004; 165(1):237e46.

In an embodiment the CCR2 inhibitor can be loaded on a nanoparticle. The nanoparticle can be any kind of nanoparticle suitable for medical use, for example lipidic and polymeric nanoparticles. The inclusion of the CCR2 inhibitor in nanoparticles can be advantageous to better control drug release and to facilitate drug cell internalization. Indeed, in this embodiment the drug release profile can be even more modulated by realizing a hierarchical structure wherein the CCR2 inhibitor is loaded on a nanoparticle and said nanoparticle is loaded in the microparticle of the invention.

The drug loading is preferably comprised between 2 and 30%, preferably between 3 and 10% per microparticle. For drug loading it is intended the amount of CCR2 inhibitor loaded in a specific mass of particles (polymer+drug). It is calculated as the fraction of drug loaded in a specific amount of particles. The amount of drug per each microparticle is usually calculated as a fraction between the mass of drug loaded in the production process and the number of microparticles.

In a preferred embodiment each microparticle comprises poly(lactic-co-glycolic acid)(PLGA) and RS504393.

The microparticle can also comprise one or more further drugs, such as for example glucocorticoids; non-steroidal anti-inflammatory drugs; monoclonal antibody against the tumor necrosis factor-alpha (TNF-α), such as certolizumab pegol; TNF-α inhibitors, such as Golimumab; antiplatelet drugs, such as Tirofiban; kinase inhibitors, such as Ruxolitinib; CLK/DYRK1A inhibitors, such as Lorecivivint; Fetuin A. Preferably, said further drug is an anti-inflammatory drug, which can advantageously reduce the pain simultaneously with the action of the CCR2 inhibitor on the cartilage and bone damage, for example it is dexamethasone.

In an embodiment the microparticle can also comprise one or more nanoparticles, such as lipidic and polymeric nanoparticles, short interfering RNA (siRNA)-loaded nanoparticles, for example against matrix metalloproteinase 13 (MMP13)). Said nanoparticles can also be loaded with one or more drugs.

The possibility to administer CCR2 inhibitor with the composition of the invention offers beneficial effects on both cartilage and bone structure at both the early and late OA stages. This was not obtained by systemic administration of free drug, which acted only in the early OA stage.

The composition of the invention can be administered with any administration route. Examples of routes of administration include parenteral, e.g. intraarticular, intradermal, subcutaneous, transdermal (topical), transmucosal, and rectal administration. Injection is a preferred administration route, intra-articularly injection is even more preferred.

The composition can be stored as a dried powder, which can be obtained by freeze-drying and water evaporation under low vacuum, as known in the field. Preferably it is stored at +4° C. until the time of administration. This storage preserves its pharmacological activity.

At the moment of the administration, the microparticles in the composition can be easily resuspended in a prefixed volume of saline solution to obtain the right dose, advantageously preventing incorrect dosage.

The drug-delivery composition of the invention is for use for the prevention and/or treatment of a localized chronic inflammation disease. For localized chronic inflammation disease is intended a disease characterized by a chronic inflammation in localized areas. Said areas are in particular joints, such as knees, hips and small joints of the hands. Preferably such disease is a chronic inflammatory joint disease, more preferably it is osteoarthritis. Osteoarthritis can be of any origin, for example it can be due to joint injury, abnormal joint or limb development, and inherited factors.

According to the present invention, preventing a disease refers to inhibiting completely, or in part, the development or progression of a disease, for example in a person who is known to have a predisposition to a disease. For example, a subject with a joint injury.

Treating a disease refers to a therapeutic intervention that ameliorates at least one sign or symptom of a disease or pathological condition, or interferes with a pathophysiological process, after the disease or pathological condition has begun to develop.

Pharmaceutically acceptable is used in the context of the present invention to refer to those compounds, materials, compositions, and/or dosage forms that are, within the scope of sound medical judgment, suitable for use in contact with the tissues of human beings and animals without excessive toxicity, irritation, allergic response, and/or other problem or complication, commensurate with a reasonable benefit/risk ratio.

The delivery of a CCR2 inhibitor with the composition of the invention through intra-articularly injection represents a great advantage because it allows to limit the therapeutic activity to the target site. This is not possible using the drug as a free form because of its solubility and its rapid clearance from knee. Furthermore, currently the CCR2 inhibitor is administered solubilized in Dimethyl sulfoxide (DMSO) that does not allow the drug injection directly in the target site. Also, the possibility to administer the drug directly in the target site reduces both the side effects connected with its systemic administration and the number and the doses of application.

Advantageously, the composition of the invention allows to combine the administration of a CCR2 receptor inhibitor directly in the target site with a sustained and controlled release of the drug.

The drug-delivery composition of the invention can be included in a pharmaceutical composition.

A pharmaceutical composition is formulated to be compatible with its intended route of administration.

In certain preferred embodiments, the pharmaceutical composition comprising at least one drug-delivery composition according to the present invention may further comprise one or more pharmaceutically acceptable carriers, exemplified by, but not limited to, lipid particles, lipid vesicles, liposomes, niosomes, sphingosomes, polymeric nanocarriers, nanoparticles, microparticles, nanocapsules, and nanospheres.

The pharmaceutical composition of the present invention is preferably in the form of a single unit dosage form that contains an amount of the therapeutic agent that is effective to treat and/or prevent an inflammatory disorder as described herein and at least one pharmaceutically acceptable excipient.

Suitable pharmaceutically acceptable excipients are those commonly known to the person skilled in the art for the preparation of compositions for intra-articular, parenteral, intradermal, subcutaneous, oral, transdermal, topical, transmucosal, and rectal administration.

By way of non-limiting example, said acceptable carriers can consists of binders, diluents, lubricants, glidants, disintegrants, solubilizing (wetting) agents, stabilizers, colorants, anti-caking agents, emulsifiers, thickeners and gelling agents, coating agents, humectants, sequestrants, and sweeteners.

The amount of the at least one drug-delivery composition in the pharmaceutical composition of the present invention will depend upon the nature and severity of the condition being treated, and on the nature of prior treatments which the patient has undergone. For a human patient, the attending physician will decide the dose of compound of the present invention with which to treat each individual patient. Initially, the attending physician can administer low doses and observe the patient's response. Larger doses may be administered until the optimal therapeutic effect is obtained for the patient, and at that point the dosage is not increased further. The duration of therapy using the pharmaceutical composition of the present invention will vary, depending on the severity of the disease being treated and the condition and potential idiosyncratic response of each individual patient.

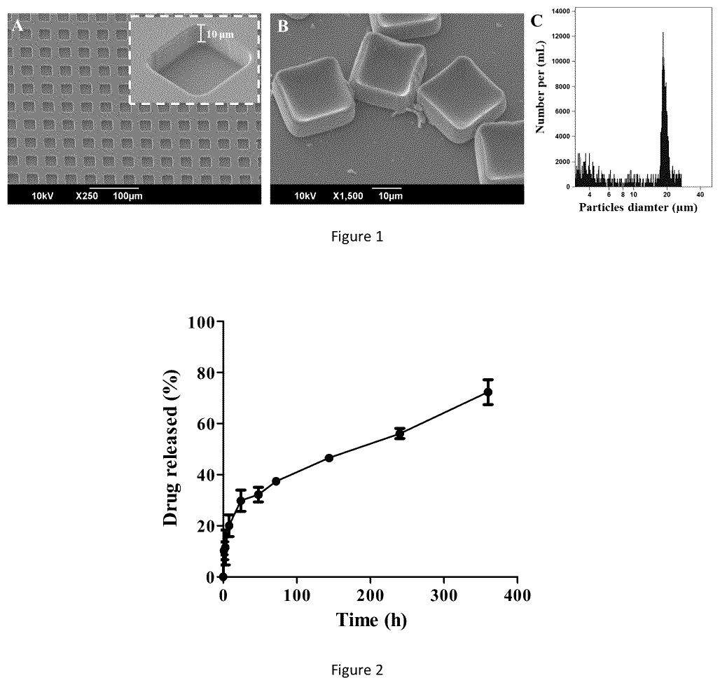

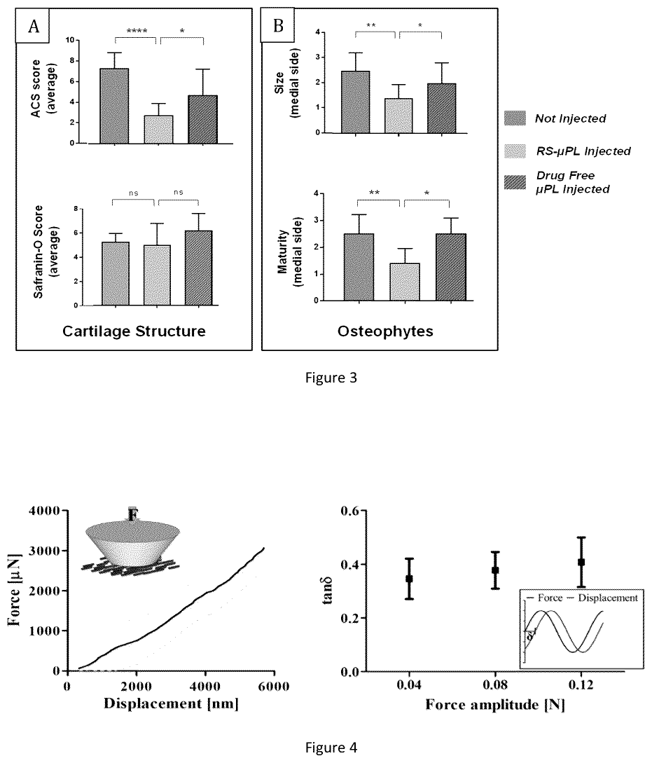

It has been shown in the experiments disclosed in the present invention that the composition of the invention can have both a good drug loading (over 15 pg per production batch corresponding to about 3% loading) and sustained and controlled release for almost 1 month, still preserving the pharmacological activity of RS504393 (see FIGS. 1, 2 and in vivo therapeutic efficacy in FIG. 3). Also, it has a mechanical damping property and exhibits an apparent Young's modulus (˜3 MPa) similar to that of cartilage, (see FIG. 4).

The following examples further illustrates the invention.

### EXAMPLES

### Example 1. μPLs Preparation

For transferring the silicon template micropattern, the poly(dimethylsiloxane) (PDMS), Sylgard™ 184 Silicone elastomer, is deposited on it and cured in oven at 60° C. for 4 hours, using a conventional ratio between the curing agent, Sylgard™ 184 silicone elastomer curing agent, and the elastomer (1:10, v/v). The latter intermediate template presents the opposite configuration of the silicon template (pillars instead of wells). Finally, a 10% w/v Poly(vinyl alcohol) (PVA) solution is spread on the PDMS template and maintained at 60° C. for promoting water evaporation. The obtained PVA template shows the geometrical pattern of the original silicon master template (wells with same size and shape). At this point, a hydrophobic polymer, poly(lactic-co-glycolic acid)(PLGA), is used for synthetizing μPLs. PLGA is dissolved in Acetonitrile with a concentration of 400 mg/mL. 15 mg of PLGA is used for each PVA template.

In this specific application, μPLs with a square shape and size of 20×20×10 μm are synthetized using an optimized amount of polymer content (15 mg, for each template) (Figure. 1). RS504393 is solubilized in warmed Acetonitrile (60° C. for 3 min) at the concentration of 1.25 mg/mL. The heating does not affect the pharmacological effect of the drug (in vivo therapeutic efficacy, FIG. 3).

### Example 2 μPLs Physico-Chemical Characterization

Scanning electron microscopy (SEM) analysis: in order to evaluate PVA template structure and individual μPLs size and shape, scanning electron microscopy (SEM, Elios Nanolab 650, FEI) was performed. Briefly, a piece of PVA template or a drop of particles was put on a silicon template and coated with gold in order to preserve samples and increase contrast. An acceleration voltage of 5-15 keV was used for SEM imaging.

Multisizer 4 COULTER particle counter analysis: μPL average size and distribution was studied using a Multisizer 4 COULTER particle counter (Beckman Coulter, CA). Particles were resuspended in electrolyte solution and analyzed, according to vendor protocols.

Encapsulation and loading efficiency: to evaluate the RS 504393 loading and encapsulation efficiency (EE and LE), particles were lyophilized, dissolved in acetonitrile/H20 (1:1, v/v), and analyzed using High Performance Liquid Chromatography (HPLC) after adding an equal volume of acetonitrile. A C18 column (2.1×100 mm, 3.5 m particle size, Agilent, USA) was used for the chromatographic separation. RS 504393 is eluted under isocratic conditions using a binary solvent system [H20+0.1% (v/v) TFA/AcN+0.1% (v/v) TFA, 43:57 v/v] pumped at a flow rate of 0.300 mL/min. The ultraviolet (UV) detection is set at 255 nm.

### Example 3 Drug Release Profile

Release study: to evaluate the RS 504393 release profile in a confined microenvironment, particles were put in three Eppendorf tubes in 500 μL of PBS buffer (pH 7.4, 1×, 37+2° C.) under continuous rotation. For each time point, samples were collected and centrifuged (1717 g for 5 min). Supernatant was analyzed using High Performance Liquid Chromatography (HPLC) after adding an equal volume of acetonitrile, as reported above.

Results are shown in FIG. 2.

The release profile of RS504393 loaded μPLs under confined microenvironment is shown in FIG. 2. In the first 48h, there was a 35% burst release and the remaining encapsulated RS 504393 was released a relatively constant rate, yielding approximately 80% release after 15 days.

### Example 4. In Vivo Efficacy Tests

RS 504393-loaded μPLs (sizes 20×20×10) are injected into the knee intra-articular space of C57BL/6 male mice undergoing destabilization of medial meniscus (DMM) surgery, a murine model of post-traumatic osteoarthritis (PTOA). Based on our release profile, we determined that exogenous administration every 3 weeks would allow a constant delivery of the antagonist into the joint. Two consecutive injections of RS-microparticles (0.5 mg/kg, in 10 μL at day 6 and day 7) were given to reach a dose of 1 mg/kg in 24 hours. Injections were repeated at week 4 and 7 post DMM. At four and ten weeks after DMM, histological coronal sections of DMM knees are prepared and stained for histological evaluation (Hematoxylin and Eosin or Safranin-O/Fast Green). Articular cartilage lesions are graded after 10 weeks post-surgery based on the appearance of the articular surface of the tibia plateau and femoral condyles (ACS score). The scale ranges from zero (articular surface smooth and intact) to 12 (fibrillation and/or cleft of the articular cartilage or, in more severe cases, loss of articular cartilage. The scale accounts for the depth of the lesions as well as for the extension on the articular surface. The Safranin-O score accounts for loss of extracellular matrix and ranges from zero (uniform staining throughout the articular cartilage) to 12 (Complete loss of staining in the cells and matrix). The scale accounts for depth of staining loss as well as for the extension of the loss. Osteophytes are graded by size, ranging from 1 (approximately same size of the adjacent cartilage), 2 (2-3 times the thickness of the adjacent cartilage) or 3 (more than 3 times the thickness of the adjacent cartilage. Osteophyte maturity is defined by the histological composition, ranging from 1 (predominantly cartilage, 2 (mix of cartilage and bone or 3 (predominantly bone).

Results are shown in FIG. 3.

Results show that intra-articular administration of RS-microparticles into DMM effectively improves articular cartilage structure compared to not injected controls (ACS score), decreasing fibrillation and articular cartilage loss, at both the early and severe OA stage (4 and 10 weeks, respectively); interestingly, blockade of the CCR2 did not significantly reduce the extracellular matrix loss (Saf-O/FG score). With respect to bone damage, we found that RS-microparticles administration significantly reduced the size of osteophytes, as well as their composition, delaying bone deposition.

These results indicate that a local continuous inhibition of the CCR2 receptor into mouse knee joints during PTOA progression has beneficial effect on both cartilage and bone structure at both the early and late OA stages. These results are different from the ones previously obtained with systemic CCR2 administration, where prolonged CCR2 blockade produced no significant differences in cartilage and bone structure at the most severe OA stages.

### Example 5. Mechanical Characterization

Mechanical characterization of μPLs: The apparent elastic modulus of μPLs was measured by flat punch micro-indentation tests. Small droplets (<1 μL) of a μPL solution were deposited over a glass slide, covering an area of ˜5 mm² with multiple particles and dried overnight. Micro-indentation was performed on an UNHT Nanoindentation platform (Anton Paar) equipped with a 200 μm-diameter truncated cone tip. Load was applied at a rate of 20 mN/min until the maximum load of 3 mN. From the slope of the force-displacement curves, the modulus was calculated through the classical Hertzian equation F=2REh where F is the applied force, h the tip displacement, R the tip radius, E the apparent elastic modulus. Three repetitions were conducted on different droplets.

The energy dissipation capability was characterized by Dynamic Mechanical Analysis (DMA) on a Q800 system (TA Instruments). Highly concentrated μPL solutions were deposited on a glass slide and partially dried in a vacuum desiccator for 10 min to create a thin layer of μPLs. Then the glass slide was transferred onto the bottom plate of a compressive clamp. A pre-load was applied gently, squeezing out excess water. A sinusoidal force was applied to the layer of μPLs with the frequency of 5 Hz and increasing amplitude (0.04, 0.08 and 0.12 N). The phase difference between the input (force) and output (deformation) was recorded as a function of the oscillation amplitude. The tangent of the phase difference angle, noted as tan δ, represents the ratio between dissipative and conservative energy during one oscillation and, as such, provides a measure of the damping capability of the material. Tests were conducted at 37° C.

Results are shown in FIG. 4.

Specifically, a small droplet (<1 μL) of a μPL solution was deposited over a glass slide, dried overnight, and indented with a 200 μm-diameter truncated cone tip. Indentation force-displacement curves were derived as shown in FIG. 4, where the average value (line) and the corresponding standard deviation (shadowed area) are presented for three repetitions. From the slope of the force-displacement curves, an apparent modulus was calculated of 3.1±0.9 MPa based on the classical Hertz theory of contact mechanics. In addition to this static characterization, dynamic testing was conducted to characterize the viscoelastic response and potential mechanical dampening behavior of μPLs. In this case, a small droplet of a μPL solution was deposited over a glass slide and partially dried in a vacuum desiccator to create a thin particle layer. Then, a sinusoidal force was applied to the μPL layer, with a frequency of 5 Hz and increasing force amplitude (0.04, 0.08 and 0.12 N). The phase difference between the input (force) and output (deformation) was recorded over time to extract the phase difference parameter (tan δ), which is related to the mechanical damping of the system. This is shown in FIG. 4 giving a tan δ of ˜0.3.

### Example 6. Evaluation of μPL Cellular Interactions

To evaluate μPL cellular interactions within ATDC5 cell line, murine chondrocytes, and phagocytic cell lines (RAW 264.7 macrophages), 20×10⁴ cells were seeded onto glass coverslips for 24 h. The cells were then incubated overnight with μPLs at a ratio of 1:4 (μPL:cells). Samples were fixed for 2 h in 2% glutaraldehyde in 0.1 M cacodylate buffer. After fixation, the samples were washed thrice with the same buffer and post fixed for 1 h in 1% osmium tetroxide in distilled water. After several washes with distilled water, the samples were subsequently dehydrated in a graded ethanol series, 1:1 ethanol:hexamethyldisilazane (HMDS), and 100% HMDS, followed by drying overnight in air. Dried samples were then mounted on stubs using silver conductive paint and coated with gold. SEM images were collected with scanning electron microscopy (SEM, Elios Nanolab 650, FEI) operating at an accelerating voltage between 5 and 15 KeV.

The inventors demonstrated the ability of μPLs to resist macrophage uptake and adhere to the cartilaginous tissue.

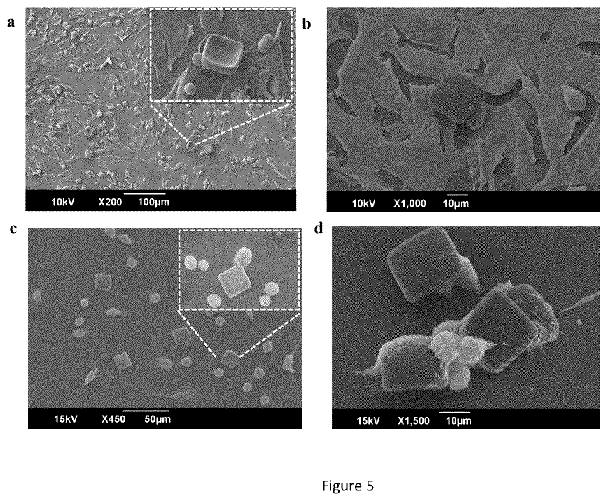

As shown in FIG. 5, μPLs are not internalized by chondrocytes and macrophages, thus remaining extracellularly in the synovial cavity and promoting drug release and mechanical support at the target site.

### Example 7. In Vivo Pharmacokinetic of Cy5 Conjugated Microplates (Cy5-μPLs)

In order to study in vivo pharmacokinetic, Cy5 conjugated microplates (Cy5-μPLs) were injected intraarticularly in PTOA model of non invasive repetitive joint loading. A single intraarticular injection of Cy5-μPLs was administered into each knee, starting concurrently with mechanical loading. A rigorous cyclic mechanical loading (on mice anesthetized with 3% isoflurane) at 9 N per load, 500 cycles per session, cycle lasting 2.5 seconds, 5 loading sessions per week, was performed for 4 weeks using a TA Electroforce 3100 (TA instruments, New Castle, Delaware, USA).

Mice were imaged intravitally for Cy5 fluorescence over time using an IVIS Lumina III intravital imaging system (Caliper Life Sciences, Hopkinton, MA). For IVIS image analysis, regions of interest (ROIs) were drawn around both the right and the left knees. For each mouse knee, a pre-injection reading (blank) was taken followed by a time 0 (TO) reading directly after intraarticular injection. The blank reading was used for background correction of all images of the same mouse knee at all time points, and dividing the radiance reading (at a specific timepoint) by the TO radiance reading was used to calculate ‘fraction of retention’ at all later timepoints. Animals were euthanized at days 1, 3, 5, 7, 10, 15, 20, 25, and 30 post-injection along with control no treatment animals (NT). At takedown, the skin was removed from the legs, and the knees were endpoint imaged ‘ex-vivo’ for Cy5 fluorescence which provides better sensitivity than intravital imaging. Organs were also harvested for Cy5 fluorescence biodistribution. After ex-vivo imaging, excess muscle was removed, and legs were then snap frozen in liquid nitrogen and stored at −80° C. until cryosectioning. For intravital imaging analysis, n=4-24 limbs depending on the time point i.e. earlier time points had more animals included as animals were taken down over time for ex-vivo and confocal microscopy analysis. For ex-vivo imaging analysis and confocal microscopy analysis, n=2-4 limbs per time point.

Legs (stored at −80° C.) were embedded into OCT freezing compound, cooled, and serial sectioned in sagittal orientation until an adequate depth of the joint was reached. Cryosections at various depths along the joint were then sectioned at 20 m thick, captured utilizing a commercially available polyvinylidene chloride film coated with synthetic rubber cement (http://section-lab.jp/), and placed on a slide. Slides were then fixed in 10% neutral buffered formalin for 5 minutes, cover slipped with an aqua mount, and imaged on a Nikon Eclipse Ti inverted confocal microscope. Imaging settings were kept constant for imaging of all Cy5-μPLs-containing joint samples at each time point (n=2-4 limbs per time point). TD=transmission detector.

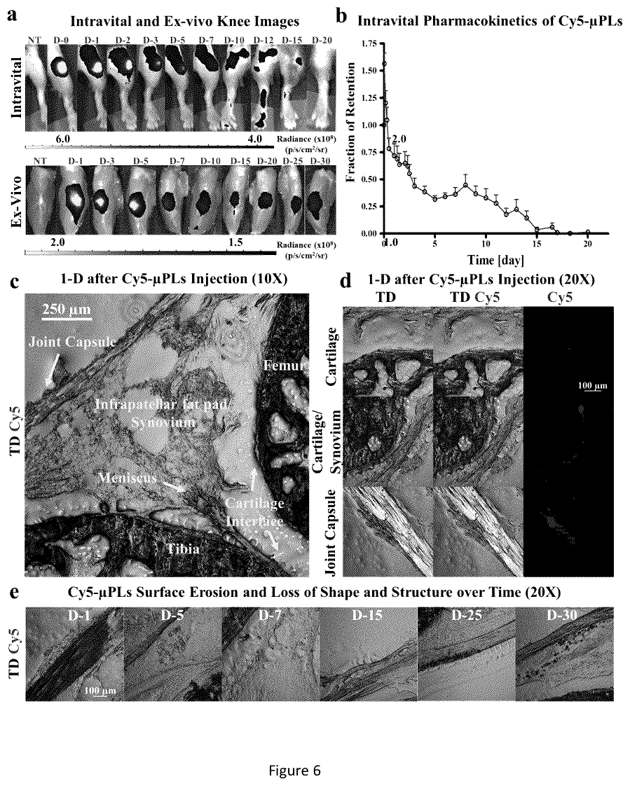

Results showed that 1 day after injection particles were dispersed across the entire joint reaching the femoral-tibial cartilage interface, the infrapatellar fat pad and synovium, and the joint capsule (FIG. 6). More specifically, μPLs were on top of the articular cartilage surface, near the cartilage/synovium interface and the joint capsule.

With this experiment inventors have proved that μPLs are retained in the knee for around 1 month after one single injection in a mouse model of post traumatic osteoarthritis (PTOA).

1: A drug-delivery composition comprising one or more microparticles, wherein each microparticle has a shape of a prism with three or more sides, and each side has a side length comprised between about 5 and about 50 μm and a height length comprised between about 5 and about 50 μm, said microparticle comprising at least one polymer and at least one inhibitor of C-C Chemokine Receptor-2 (CCR2).

2: The drug-delivery composition according to claim 1 wherein said microparticle has the shape of a prism with a square base.

3: The drug-delivery composition according to claim 2 wherein the side length is of about 20 μm and the height length is of about 10 μm.

4: The drug-delivery composition of claim 1, wherein said microparticle has a Young's modulus under compression comprised between 1 KPa and 10 MPa, preferably from 100 KPa to 10 MPa.

5: The drug-delivery composition of claim 1, wherein said polymer is selected from the group consisting of poly(lactic-co-glycolic acid)(PLGA), polyethylene glycol (PEG), polycaprolactone (PCL), hyaluronic acid (HA), chitosan, gelatin and a combination thereof.

6: The drug-delivery composition of claim 1, wherein said CCR2 inhibitor is selected from the group consisting of RS504393, Maraviroc, cenicriviroc, CD192, CCX872, CCX140, 2-((Isopropylaminocarbonyl)amino)-N-(2-((cis-2-((4(methylthio)benzoyl)amino)cyclohexyl)amino)-2-oxoethyl)-5-(trifluoromethyl)-benzamide, vicriviroc, SCH351125, TAK779 and CCR2 antagonist 4.

7: The drug-delivery composition of claim 1, wherein said CCR2 inhibitor is loaded on a nanoparticle.

8: The drug-delivery composition of claim 1, wherein each microparticle comprises poly(lactic-co-glycolic acid)(PLGA) and RS504393.

9: The drug-delivery composition of claim 1, wherein said microparticle further comprises an anti-inflammatory drug, or dexamethasone.

10: The drug-delivery composition of claim 1, wherein said microparticle further comprises a drug selected from the group consisting of: glucocorticoids; non-steroidal anti-inflammatory drugs; monoclonal antibody against the tumor necrosis factor-alpha (TNF-α), certolizumab pegol; TNF-α inhibitors, Golimumab; antiplatelet drugs, Tirofiban; kinase inhibitors, such as Ruxolitinib; CLK/DYRK1A inhibitors, Lorecivivint; and Fetuin A.

11: The drug-delivery composition of claim 1, wherein said microparticle further comprises one or more nanoparticles, lipidic and polymeric nanoparticles or short interfering RNA (siRNA)-loaded nanoparticles.

12: The drug-delivery composition of claim 1, for use as a medicament.

13: A method for treating or preventing a localized chronic inflammation disease comprising administering to an individual in need thereof a drug-delivery composition of claim 1.

14: The method of claim 13, wherein said localized chronic inflammation disease is osteoarthritis.

15: The method of claim 13 wherein said composition is administered by intra-articular injection.

16: A pharmaceutical composition comprising: the drug delivery composition of claim 1, and (b) a pharmaceutically acceptable excipient and/or a carrier.

17: A drug-delivery composition comprising one or more microparticles, wherein each microparticle has non-spherical shape, and each microparticle is between about 1 and 100 μm in size, and said microparticle comprises at least one polymer and at least one inhibitor of C-C Chemokine Receptor-2 (CCR2).

18: The drug-delivery composition of claim 17, wherein each microparticle is in the shape of a prism.

19: The drug-delivery composition of claim 17, wherein the prism is a polyhedron comprising a first n-sided polygonal base, a second base which is a translated copy that is rigidly moved without rotation, and n other faces joining corresponding sides of the two bases, and the n other faces are parallelograms, wherein n is the number of sides of the polyhedron.

20: The drug-delivery composition of claim 19, wherein n is between about 3 and 8, or is 4.
